SEQUENCE LISTING (14 sequences)

SEQ ID NO: 1 - DNA, 1800 bp, Homo sapiens
  source 1..1800: mol_type = other DNA
atggcgaccaacggcagcaaggtggccgacgggcagatctccaccgaggtcagcgaggcccctgtggccaatgacaagcccaaaaccttggtggtcaaggtgcagaagaaggcggcagacctccccgaccgggacacgtggaagggccgcttcgacttcctcatgtcctgtgtgggctatgccatcggcctgggcaacgtctggaggttcccctatctctgcgggaaaaatggtgggggagccttcctgatcccctatttcctgacactcatctttgcgggggtcccactcttcctgctggagtgctccctgggccagtacacctccatcggggggctaggggtatggaagctggctcctatgttcaagggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctag

SEQ ID NO: 2 - protein, 599 aa, Homo sapiens [sequence omitted]

SEQ ID NO: 3 - DNA, 3140 bp, synthetic construct
  misc_feature 1..3140: note = Synthetic polynucleotide
  misc_feature 1..106: note = MISC_Feature - Mut ITR
  misc_feature 136..421: note = MISC_Feature - CMV Enhancer
  misc_feature 422..691: note = MISC_Feature - CB promoter
  misc_feature 756..855: note = MISC_Feature - Intron
  misc_feature 921..2720: note = MISC_Feature - SLC6A1
  misc_feature 2774..2920: note = MISC_Feature - BGhpA
  misc_feature 3000..3140: note = MISC_Feature - 3' ITR
  source 1..3140: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcaattcacgcgccggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtcttttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtatggcgaccaacggcagcaaggtggccgacgggcagatctccaccgaggtcagcgaggcccctgtggccaatgacaagcccaaaaccttggtggtcaaggtgcagaagaaggcggcagacctccccgaccgggacacgtggaagggccgcttcgacttcctcatgtcctgtgtgggctatgccatcggcctgggcaacgtctggaggttcccctatctctgcgggaaaaatggtgggggagccttcctgatcccctatttcctgacactcatctttgcgggggtcccactcttcctgctggagtgctccctgggccagtacacctccatcggggggctaggggtatggaagctggctcctatgttcaagggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctagaagcttgatatcctcgaggctagcggatccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 4 - DNA, 3102 bp, synthetic construct
  misc_feature 1..3102: note = Synthetic polynucleotide
  misc_feature 1..106: note = MISC_Feature - Mut ITR
  misc_feature 142..687: note = MISC_Feature - 546 promoter
  misc_feature 718..816: note = MISC_Feature - Intron
  misc_feature 883..2682: note = MISC_Feature - SLC6A1
  misc_feature 2736..2882: note = MISC_Feature - BGhpA
  misc_feature 2962..3102: note = MISC_Feature - 3' ITR
  source 1..3102: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcaattcacgcgccggtaccgaattcacgcgtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaacgcgtcgaagggcgaattctgcagataactggtaagtttagtcttttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtatggcgaccaacggcagcaaggtggccgacgggcagatctccaccgaggtcagcgaggcccctgtggccaatgacaagcccaaaaccttggtggtcaaggtgcagaagaaggcggcagacctccccgaccgggacacgtggaagggccgcttcgacttcctcatgtcctgtgtgggctatgccatcggcctgggcaacgtctggaggttcccctatctctgcgggaaaaatggtgggggagccttcctgatcccctatttcctgacactcatctttgcgggggtcccactcttcctgctggagtgctccctgggccagtacacctccatcggggggctaggggtatggaagctggctcctatgttcaagggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctagaagcttgatatcctcgaggctagcggatccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 5 - DNA, 3210 bp, synthetic construct
  misc_feature 1..3210: note = Synthetic polynucleotide
  misc_feature 1..106: note = MISC_Feature - mITR
  misc_feature 142..794: note = MISC_Feature - gfABC1D promoter
  misc_feature 826..925: note = MISC_Feature - Intron
  misc_feature 991..2790: note = MISC_Feature - SLC6A1
  misc_feature 2844..2990: note = MISC_Feature - BGhpA
  misc_feature 3070..3210: note = MISC_Feature - 3' ITR
  source 1..3210: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcaattcacgcgccggtaccgaattcacgcgtggtaccaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccatccggaacgcgtcgaagggcgaattctgcagataactggtaagtttagtcttttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtatggcgaccaacggcagcaaggtggccgacgggcagatctccaccgaggtcagcgaggcccctgtggccaatgacaagcccaaaaccttggtggtcaaggtgcagaagaaggcggcagacctccccgaccgggacacgtggaagggccgcttcgacttcctcatgtcctgtgtgggctatgccatcggcctgggcaacgtctggaggttcccctatctctgcgggaaaaatggtgggggagccttcctgatcccctatttcctgacactcatctttgcgggggtcccactcttcctgctggagtgctccctgggccagtacacctccatcggggggctaggggtatggaagctggctcctatgttcaagggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctagaagcttgatatcctcgaggctagcggatccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 6 - DNA, 3041 bp, synthetic construct
  misc_feature 1..3041: note = Synthetic polynucleotide
  misc_feature 1..106: note = MISC_feature - Mut ITR
  misc_feature 142..626: note = MISC_feature - hSYN promoter
  misc_feature 657..756: note = MISC_feature - Intron
  misc_feature 822..2621: note = MISC_feature - SLC6A1
  misc_feature 2675..2821: note = MISC_feature - BGhpA
  misc_feature 2901..3041: note = MISC_feature - 3' ITR
  source 1..3041: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcaattcacgcgccggtaccgaattcacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaacgcgtcgaagggcgaattctgcagataactggtaagtttagtcttttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtatggcgaccaacggcagcaaggtggccgacgggcagatctccaccgaggtcagcgaggcccctgtggccaatgacaagcccaaaaccttggtggtcaaggtgcagaagaaggcggcagacctccccgaccgggacacgtggaagggccgcttcgacttcctcatgtcctgtgtgggctatgccatcggcctgggcaacgtctggaggttcccctatctctgcgggaaaaatggtgggggagccttcctgatcccctatttcctgacactcatctttgcgggggtcccactcttcctgctggagtgctccctgggccagtacacctccatcggggggctaggggtatggaagctggctcctatgttcaagggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctagaagcttgatatcctcgaggctagcggatccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 7 - DNA, 2919 bp, synthetic construct
  misc_feature 1..2919: note = Synthetic polynucleotide
  misc_feature 1..106: note = MISC_feature - Mut ITR
  misc_feature 148..497: note = MISC_Feature - SST promoter
  misc_feature 535..634: note = MISC_Feature - Intron
  misc_feature 700..2499: note = MISC_Feature - SLC6A1
  misc_feature 2552..2699: note = MISC_Feature - BGhpA
  misc_feature 2779..2919: note = MISC_Feature - 3' ITR
  source 1..2919: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcaattcacgcgccggtaccgaattcacgcgtggtaccgcatgtgtgggagtgaaattatggaatgtgtatgctcatagcactgagtgaaaataaaagattgtataaatcgtggggcttgtggaattgtgagtccctgtgcgtgtgcagtatttttttttttttttaagtaagactctttagatcttgtcgcctcccctgtcttctgtgattgattttgcgagactaatggtgcgtaaaagggctggtgagatctgggggcgcctcctagcctgacgtcagagagagagtttaaaaccgagggagacggttgagagcacacaagccgctttaggagtcgcgaggttcggagccatcgctgctgcctgctgatccgcgcctagagtttgtccggaacgcgtcgaagggcgaattctgcagataactggtaagtttagtcttttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtatggcgaccaacggcagcaaggtggccgacgggcagatctccaccgaggtcagcgaggcccctgtggccaatgacaagcccaaaaccttggtggtcaaggtgcagaagaaggcggcagacctccccgaccgggacacgtggaagggccgcttcgacttcctcatgtcctgtgtgggctatgccatcggcctgggcaacgtctggaggttcccctatctctgcgggaaaaatggtgggggagccttcctgatcccctatttcctgacactcatctttgcgggggtcccactcttcctgctggagtgctccctgggccagtacacctccatcggggggctaggggtatggaagctggctcctatgttcaagggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctagaagcttgatatcctcgaggctagcggatccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 8 - DNA, 4770 bp, synthetic construct
  misc_feature 1..4770: note = Synthetic polynucleotide
  misc_feature 1..106: note = MISC_Feature - ITR
  misc_feature 200..346: note = MISC_Feature - BGhpA
  misc_feature 400..2199: note = MISC_Feature - SLC6A1
  misc_feature 2266..2363: note = MISC_Feature - Intron
  misc_feature 2396..4609: note = MISC_Feature - GFAP promoter
  misc_feature 4630..4770: note = MISC_Feature - ITR
  source 1..4770: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgacggatccgctagcctcgaggatatcaagcttctagatgtaggcctccttgctggtggagctgcccgcctggggctgctcaggaccattctctgggcgaacgatgtcttcgctgggctggaccatgacttggatgcgctgcttcagggagccctttaaggtgaggaacatgtaggccatgtacccggggatgaggaccatggaagacagagccatcagccagcccacaccctggccccacttggggaaaacatagtttcccatggtgagtggcgtcatctgcacagcactgaaaatgaacacgcccgccacaatgattggtgtgaagaaagaccagcagagtttccaccagatgcagggcctggatccaaccatctcttggatattgtcatagaatcggttgacaccgtaaaaccaggaaatagagacacattcaaagaacacgaggaacagcaggctcatgccactggcagagtagtagtcaaagagtttgaagacataaatacccccctgagtgatgttagagagaccgatcaggtaggagatgatgcagacagcagcaatgaagagctctctgcggttgcggaggagcctggggtactcatccaccagggctgtgatgaagccctccacagtgcagaactggctgtcaatgcccagcatcaacagcatggagaagaagaggatggcccagagtggggagataggcagctgggtcaccgcctctgggtatgccaggaacgccagcccggggcctgaggccgccacatcagcaatggacctcttggtgacatgggccatgaagcccacgatggagaagatgacgaatcctgcgaacatgctggtgcacgaattgatgcagcagacgatgatggagtccctgtagacattgttgtggaaagagttgtagctcccgagagcgatcagggaccccaggcccagcccgtatgagaagaagatctgggttgccgcatccagccacacctcggagtcagacagcttgcggaagttgggtgtgatgtagaagaggatgccctccttggccccgggcagcgtcactccacggaagaacaggatgatcagcatgatgtaggggtatgtggctgaaaagtagaccacctttccagtccagccaacacccttccagatacagaaatacacaaggatccaggcgatggccagcgtgatggccagtggccagcggatctgacctggcttatccagcccgtccgtcatctgatgcatgttgcgctcccagaactccaccacagcgctggtcatgttggtagtgttgaccatgctgtagttggagaagcagcggtctgtgttccaggggttgtcgcactgtttccacggcagtgtcgtggtgaaggagttgtacaggtagtaaatggcccaggagatgatgacgatgtagtagatgttcagccagaatgatagcacagcagccgcaaggcccacgcccttgaacataggagccagcttccatacccctagccccccgatggaggtgtactggcccagggagcactccagcaggaagagtgggacccccgcaaagatgagtgtcaggaaataggggatcaggaaggctcccccaccatttttcccgcagagataggggaacctccagacgttgcccaggccgatggcatagcccacacaggacatgaggaagtcgaagcggcccttccacgtgtcccggtcggggaggtctgccgccttcttctgcaccttgaccaccaaggttttgggcttgtcattggccacaggggcctcgctgacctcggtggagatctgcccgtcggccaccttgctgccgttggtcgccataccggtggatcggccgcgggtacaattccgcagcttttagagcagaagtaacacttccgtacaggcctagaagtaaaggcaacatccactgaggagcagttctttgatttgcaccaccaccggatccgggacctgaaataaaagacaaaaaagactaaacttaccagttatctgcagaattcgcccttcgacgcgtccggcgagcagcggaggtgatgcgtctcctctccaacctgctctggctctgctcgctcctgggatgcgagggctttatgaaggagtgggctagactggcgatgcccgggtgcccctggcaacaccccctggcatagcctgagggggtggggttgggccgcagcccagctatggggagagctcccctcaccccactgaggtcactgtgcccagaggcagagcttcctgggcacgccaactcctgcatgaggcaggtggtacctggagccagaccatgggtctggggccctgaccgctgaagcaagaggaccctctctctaggaaggtgggtcaagaaagggttggttggaccatgatgaatacttacaattattatgcatcaattaaaaaggaaggaaagggctgagtggggtggctcatgcttgtaatcccagcactttgggaggctgaggctggaggattgcttgagcccagtaggttgaggctgcagtgagctgtgtttgcgccactccagcctgggcaacagagagagaccttgtctcacaaaaaaaaaaaaaaaaaaagggaaggaagggctgtaccagatgacctctgacctgctttccaactttttgatttcataacccaggcattatctcactcagctgtaagtgcctgcaaggtgctgggaatttggcgaagaatgaacctgttctgtaccctcaagggccacctcatttcctactaggcctcattcccagcagactcctggagtccttcccttgaacacagatgtcccacattcctgctctctgcccattcattccctggagttctggggctgggggctgggattggacaccagccctttgtctaagggtcaatcatggcctctatgtggcagtggaggtcctgatagggcaagagggggccggcactctcttgccttatcaggactttagcccctggatctgtccctccaggtcaccccttccttgtctggcctccctgtgcctctctcccagagaccaggccttgacagctccacaaagccgttacacatgagctggctgtgctggctcgtctttattcctgcagcgccccgtttccctggccacccctccgtctcccactggagatttatttcctcgagttcccacacatcagcctggagagatgggaagaacctctggggatggatgggtttggaaacgctggagccactgggcccaactaggagcctgagacctgggccctgttgtccctggagacttcaggtccatctacgccctccactctggtcccacagggtcagaaccctgcccagtcccctcacccatttgtgtccttggacttctgggaagcagacagccctcctctccccccctgccacccactgtccatgcctgggcatcccctgatcccctttcctgggcacaggctgaatagagccttgttctccaccgccggtggcgccacacagcaagggcagcccctagggggctgtgctgcttttatcccaagatgccaggctgtcaggctggggtgcaatgcaagccccctgctcaatgggcttctcggaaggcatttgaggggggctggggtgcggcgggaagcaggcactgtgcccaccccgaacattgtgtctgtgccaaggtgagtcattcactgggcatgaagaggaggccctggggggcggccagcccaatgctgccaggtctgcttgcagacaaggcctggctgtctgcctcctccccagctcacagagccagctcaggcccccgagcctctgtcagagcagcgtcccctactccacaccaggatatgttctttttttttttttttttttttttgtcttttgagacagggtctcactgttgcccaggctggagtgcagtggcgcaaccacgactcactgcagcctcaacctcccagccttaagtgatcccctcacctcagcctcctgaatagctgggactacaggtgtgtgtcaccacacgtggctgattttttaactttttttgtagagatagggtcttgccatgttgcccaggctggtctagaacttctgggctcaagtgatccttccacatcagcctcccaaagtgctggaattacagttgtgcgccactgcgcccagcccagggtatgctctgcatttggacatgtgatggacagactgcctcctgaggtctccctctgtgagtcccagcacagagagggaggtgggacgcgtgaattcggtaccgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 9 - DNA, 270 bp, synthetic construct
  misc_feature 1..270: note = Synthetic polynucleotide
  source 1..270: mol_type = other DNA
ccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggag

SEQ ID NO: 10 - DNA, 546 bp, synthetic construct
  misc_feature 1..546: note = Synthetic polynucleotide
  source 1..546: mol_type = other DNA
gaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaa

SEQ ID NO: 11 - DNA, 653 bp, synthetic construct
  misc_feature 1..653: note = Synthetic polynucleotide
  source 1..653: mol_type = other DNA
ggtaccaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccatccgga

SEQ ID NO: 12 - DNA, 485 bp, synthetic construct
  misc_feature 1..485: note = Synthetic polynucleotide
  source 1..485: mol_type = other DNA
gtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaa

SEQ ID NO: 13 - DNA, 350 bp, synthetic construct
  misc_feature 1..350: note = Synthetic polynucleotide
  source 1..350: mol_type = other DNA
gcatgtgtgggagtgaaattatggaatgtgtatgctcatagcactgagtgaaaataaaagattgtataaatcgtggggcttgtggaattgtgagtccctgtgcgtgtgcagtatttttttttttttttaagtaagactctttagatcttgtcgcctcccctgtcttctgtgattgattttgcgagactaatggtgcgtaaaagggctggtgagatctgggggcgcctcctagcctgacgtcagagagagagtttaaaaccgagggagacggttgagagcacacaagccgctttaggagtcgcgaggttcggagccatcgctgctgcctgctgatccgcgcctagagtttg

SEQ ID NO: 14 - DNA, 2214 bp, synthetic construct
  misc_feature 1..2214: note = Synthetic polynucleotide
  source 1..2214: mol_type = other DNA
ccggcgagcagcggaggtgatgcgtctcctctccaacctgctctggctctgctcgctcctgggatgcgagggctttatgaaggagtgggctagactggcgatgcccgggtgcccctggcaacaccccctggcatagcctgagggggtggggttgggccgcagcccagctatggggagagctcccctcaccccactgaggtcactgtgcccagaggcagagcttcctgggcacgccaactcctgcatgaggcaggtggtacctggagccagaccatgggtctggggccctgaccgctgaagcaagaggaccctctctctaggaaggtgggtcaagaaagggttggttggaccatgatgaatacttacaattattatgcatcaattaaaaaggaaggaaagggctgagtggggtggctcatgcttgtaatcccagcactttgggaggctgaggctggaggattgcttgagcccagtaggttgaggctgcagtgagctgtgtttgcgccactccagcctgggcaacagagagagaccttgtctcacaaaaaaaaaaaaaaaaaaagggaaggaagggctgtaccagatgacctctgacctgctttccaactttttgatttcataacccaggcattatctcactcagctgtaagtgcctgcaaggtgctgggaatttggcgaagaatgaacctgttctgtaccctcaagggccacctcatttcctactaggcctcattcccagcagactcctggagtccttcccttgaacacagatgtcccacattcctgctctctgcccattcattccctggagttctggggctgggggctgggattggacaccagccctttgtctaagggtcaatcatggcctctatgtggcagtggaggtcctgatagggcaagagggggccggcactctcttgccttatcaggactttagcccctggatctgtccctccaggtcaccccttccttgtctggcctccctgtgcctctctcccagagaccaggccttgacagctccacaaagccgttacacatgagctggctgtgctggctcgtctttattcctgcagcgccccgtttccctggccacccctccgtctcccactggagatttatttcctcgagttcccacacatcagcctggagagatgggaagaacctctggggatggatgggtttggaaacgctggagccactgggcccaactaggagcctgagacctgggccctgttgtccctggagacttcaggtccatctacgccctccactctggtcccacagggtcagaaccctgcccagtcccctcacccatttgtgtccttggacttctgggaagcagacagccctcctctccccccctgccacccactgtccatgcctgggcatcccctgatcccctttcctgggcacaggctgaatagagccttgttctccaccgccggtggcgccacacagcaagggcagcccctagggggctgtgctgcttttatcccaagatgccaggctgtcaggctggggtgcaatgcaagccccctgctcaatgggcttctcggaaggcatttgaggggggctggggtgcggcgggaagcaggcactgtgcccaccccgaacattgtgtctgtgccaaggtgagtcattcactgggcatgaagaggaggccctggggggcggccagcccaatgctgccaggtctgcttgcagacaaggcctggctgtctgcctcctccccagctcacagagccagctcaggcccccgagcctctgtcagagcagcgtcccctactccacaccaggatatgttctttttttttttttttttttttttgtcttttgagacagggtctcactgttgcccaggctggagtgcagtggcgcaaccacgactcactgcagcctcaacctcccagccttaagtgatcccctcacctcagcctcctgaatagctgggactacaggtgtgtgtcaccacacgtggctgattttttaactttttttgtagagatagggtcttgccatgttgcccaggctggtctagaacttctgggctcaagtgatccttccacatcagcctcccaaagtgctggaattacagttgtgcgccactgcgcccagcccagggtatgctctgcatttggacatgtgatggacagactgcctcctgaggtctccctctgtgagtcccagcacagagagggaggtgggacgcgt

(19) United States
(12) Patent Application Publication
(10) Pub. No.: US 2025/0002939 A1
(43) Pub. Date: Jan. 2, 2025

(54) MATERIALS AND METHODS FOR SLC6A1 GENE THERAPY

(71) Applicant: RESEARCH INSTITUTE AT NATIONWIDE CHILDREN'S HOSPITAL, Columbus, OH (US)

(72) Inventors: Kathrin Christine Meyer, Columbus, OH (US); Allison Marie Bradbury, Columbus, OH (US); Shibi Likhite, Columbus, OH (US)

(21) Appl. No.: 18/708,777

(22) PCT Filed: Nov. 11, 2022
(86) PCT No.: PCT/US22/79756

§ 371 Date: May 9, 2024

Related U.S. Application Data
(60) Provisional application No. 63/278,905, filed on Nov. 12, 2021.

(51) Int. Cl. C12N 15/86; A61K 38/17; A61K 48/00; C07K 14/47

(52) U.S. Cl. CPC C12N 15/86; A61K 38/1709; A61K 48/005; C07K 14/47; C12N 2750/14143

(57) ABSTRACT

The present disclosure relates to methods of treating conditions associated with a need for the electrogenic sodium- and chloride-coupled y-aminobutyric acid transporter (GAT-1) protein. for example due to a defective SLC6A1 gene as in pediatric epileptic encephalography. In particular. the disclosure provides gene therapy vectors to specifically treat loss of expression of the GAT-1 protein and/or reduced GAT-1 protein levels.

### CROSS-REFERENCE TO RELATED APPLICATIONS

This application claims priority to U.S. Provisional Patent Application No. 63/278,905, filed on Nov. 12, 2021, which is incorporated by reference herein in its entirety.

### FIELD

The present disclosure relates to methods of treating conditions associated with a need for the electrogenic sodium- and chloride-coupled γ-aminobutyric acid transporter (GAT-1) protein, for example due to a defective SLC6A1 gene as in pediatric epileptic encephalography. In particular, the disclosure provides gene therapy vectors to specifically treat loss of expression of the GAT-1 protein and/or reduced GAT-1 protein levels.

### INCORPORATION BY REFERENCE OF THE SEQUENCE LISTING

This application contains, as a separate part of disclosure, a Sequence Listing in computer-readable form (Filename: 56330_Seqlisting.XML; 59,174 bytes;—ASCII text file created Nov. 8, 2022) which is incorporated by reference herein in its entirety.

### BACKGROUND

The SLC6A1 gene encodes the electrogenic sodium- and chloride-coupled γ-aminobutyric acid (GABA) transporter, GAT-1. The GAT-1 protein localizes to the plasma membrane in GABAergic neurons and astrocytes. There it is responsible for the reuptake of the inhibitory neurotransmitter GABA from the synapse, that is removal of GABA from the synaptic cleft.

Autosomal dominant mutations in the SLC6A1 gene result in a form of pediatric epileptic encephalopathy. Primary symptoms comprise various forms of seizures, impaired cognitive development, and ataxia.

The majority of genetic variants arise de novo, and the functional effects of a number of mutations have recently been confirmed. A study of 460 epilepsy patients identified eight patients with disease-associated SLC6A1 variants including five missense mutations, one nonsense, one splice-site, and one in-frame deletion. Induction of the identified SLC6A1 variants into the rat GAT-1 sequence resulted in a range from complete abolishment to a reduction up to 27% of wild-type activity GABA transport activity [Mattison et al., SLC6A1 variants identified in epilepsy patients reduce gamma-aminobutyric acid transport, Epilepsia, 59(9):e135-e41 (2018)].

Treatment of patients is currently limited to symptomatic treatment, primarily by use of antiepileptic drugs. These drugs do not address the underlying genetic defect and thus offer no hope of stopping or slowing disease progression and when given for long durations can result in loss of efficacy.

There thus remains a need in the art for treatments for conditions, including pediatric epileptic encephalopathy, in which there is a need for GAT-1 protein GABA transporter activity.

### SUMMARY

The disclosure provides gene therapy vectors that express a functional GAT-1 protein. The gene therapy vectors are useful for delivering a transgene encoding a GAT-1 protein to a subject in need of GAT-1 GABA transporter activity (i.e., activity that removes GABA from the synaptic cleft).

The provided methods treat conditions involving reduced GAT-1 protein levels. Such conditions include, but are not limited to, pediatric epileptic encephalopathy.

The disclosure provides methods of treatment comprising delivering the gene therapy vectors to the cerebrospinal fluid (CSF) of a subject via intracerebroventricular injection, cisternal injection or lumbar intrathecal injection, or other injection method(s) accessing the CSF, or via intravenous delivery, or via a combination of such routes.

The gene therapy vector is administered to a subject in need thereof, for example, using intrathecal delivery and the subject is placed in the Trendelenburg position after administration of the gene therapy vector.

The gene therapy vectors are useful for delivering a transgene to GABAergic neurons and/or astrocytes with reduced GAT-1 protein levels in a subject.

The gene therapy vector is, for example, AAV1, AAV2, AAV3, AAV4, AAV5, AAV6, AAV7, AAV8, AAV9, AAV10, AAV11, AAV12, AAV13, AAVTT, Anc80, AAV-7m8, Anc80L65, AAVRH10, AAVRH74, or AAV-B1, or a derivative of any of these. The gene therapy vector is, for example, AAV2, AAV5, AAV6, AAV9, AAV8 or AAV10. The gene therapy vector is, for example, AAV9, AAV8 or AAV10. The gene therapy vector is, for example, AAV2, AAV5 or AAV6.

The transgene in the gene therapy vector comprises, for example, a promoter that drives expression in neurons and astrocytes of the GAT-1 protein with GABA transporter activity.

The gene therapy vector comprises, for example, a SLC6A1 cDNA.

### BRIEF DESCRIPTION OF THE DRAWINGS

FIG. 1 shows exemplary transgenes flanked by AAV ITRs for expression of GAT-1 protein from a SLC6A1 cDNA.

FIG. 2 shows expression in HEK293 cells of mRNA from rAAV comprising transgenes of FIG. 1.

FIG. 3A-E shows expression in wild type mice of mRNA from rAAV comprising transgenes of FIG. 1.

FIG. 4A-B shows body weights of treated S295L mice.

FIG. 5A-D shows results of behavior tests of treated S295L mice: A, Rotarod day 40; B, Cage hanging day 40; D, Cage hanging day 140; and E, Cage hanging day 140.

FIG. 6 shows results of clasping tests of treated S295L mice.

FIG. 7A-C shows results of behavior tests of additional mice.

FIG. 8 shows the results of EEG tests of treated S295L mice.

### DETAILED DESCRIPTION

### Gene Therapy Vectors

Adeno-associated virus (AAV) is an example of a gene therapy vector. It is a replication-deficient parvovirus, the single-stranded DNA genome of which is about 4.7 kb in length including two 145 nucleotide inverted terminal repeats (ITRs) and may be used to refer to the virus itself or derivatives thereof. The term covers all subtypes and both naturally occurring and recombinant forms, except where specified otherwise. There are multiple serotypes of AAV. The serotypes of AAV are each associated with a specific clade, the members of which share serologic and functional similarities. Thus, AAVs may also be referred to by the clade. For example, AAV9 sequences are referred to as “clade F” sequences (Gao et al., J. Virol., 78: 6381-6388 (2004). The present disclosure contemplates the use of any sequence within a specific clade, e.g., clade F. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. The sequence of the AAV rh.74 genome is provided in see U.S. Pat. No. 9,434,928, incorporated herein by reference. The sequence of the AAV-B1 genome is provided in Choudhury et al., Mol. Ther., 24(7): 1247-1257 (2016). The sequence of Anc80 is provided in Zinn et al., Cell Reports 12: 1056-1068, 2015 and Vandenberghe et al, PCT/US2014/060163, both of which are incorporated by reference herein, in their entirety and GenBank Accession Nos. KT235804-KT235812.

Cis-acting sequences directing viral DNA replication, encapsidation/packaging, and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The native AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins can be provided in trans. Another significant feature of AAV is that it is an extremely stable and hardy virus. It easily withstands the conditions used to inactivate adenovirus (56° to 65° C. for several hours), making cold preservation of AAV less critical. AAV may even be lyophilized. Finally, AAV-infected cells are not resistant to superinfection.

The term “AAV” as used herein refers to the wild type AAV virus or viral particles. The terms “AAV,” “AAV virus,” and “AAV viral particle” are used interchangeably herein. The term “rAAV” refers to recombinant, infectious, encapsidated virus or viral particles. The terms “rAAV,” “rAAV virus,” and “rAAV viral particle” are used interchangeably herein.

The term “rAAV genome” refers to a polynucleotide sequence that is derived from a native AAV genome that has been modified. rAAV genomes are provided that have been modified to remove the native AAV cap and rep genes. The rAAV genomes comprise at least one or both endogenous 5′ and 3′ inverted terminal repeats (ITRs). The rAAV genome can comprise ITRs from an AAV serotype that is different from the AAV serotype from which the AAV genome was derived. The rAAV genome can comprise three ITRs (e.g., as in scAAV).

rAAV genomes comprising a transgene flanked at the 5′ and 3′ ends by AAV ITRs are provided herein.

SEQ ID NO: 1 sets out the polynucleotide sequence of a SLC6A1 cDNA. SEQ ID NO: 2 sets out the amino acid sequence of the GAT-1 protein encoded by SEQ ID NO: 1.

Transgenes provided herein include, but are not limited to, a transgene comprising the SLC6A1 cDNA, or a polynucleotide encoding a GAT-1 protein with GABA transporter activity wherein the polynucleotide is 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the polynucleotide of SEQ ID NO: 1.

Transgenes provided herein include, but are not limited to, the transgenes set out in SEQ ID NOs: 3, 4, 5, 6, 7 and 8 (these SEQ ID NOs also each include a 5′ and a 3′ AAV ITR flanking the transgene) which each comprise the SLC6A1 cDNA of SEQ ID NO: 1. Also provided herein are transgenes encoding a GAT-1 protein with GABA transporter activity, wherein the transgenes are at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 3, 4, 5, 6, 7 or 8.

Transgenes provided herein can encode, for example, a GAT-1 protein with GABA transporter activity that is at least about: 90%, 91%, 92%, 93%, 94%, 95%, 96%, 97%, 98%, or 99% identical to the GAT-1 protein of SEQ ID NO: 2.

Transgenes provided herein include a polynucleotide that encodes a GAT-1 protein with GABA transporter activity and that hybridizes under stringent conditions to a transgene comprising SEQ ID NO: 1 or to a transgene of SEQ ID NO: 3, 4, 5, 6, 7 or 8, or the complement thereof.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing include but are not limited to 0.015 M sodium chloride, 0.0015 M sodium citrate at 65-68° C. or 0.015 M sodium chloride, 0.0015M sodium citrate, and 50% formamide at 42° C. See, for example, Sambrook et al., Molecular Cloning: A Laboratory Manual, 2nd Ed., Cold Spring Harbor Laboratory, (Cold Spring Harbor, N.Y. 1989).

Examples of promoters are the chicken β actin promoter (CBA) (SEQ ID NO: 9), a truncated methyl CpG binding protein 2 (MeCP2) promoter call the P546 MeCP2 promoter (SEQ ID NO: 10) (for driving expression in, for example, neurons and astrocytes), the human synapsin (hSyn) promoter (SEQ ID NO: 12) (for driving expression, for example, in neurons), the human somatostatin (hSST) promoter (SEQ ID NO: 13) (for driving expression, for example, in inhibitory neurons), the compact glial fibrillary acidic protein [gfaABC(1)D] promoter (SEQ ID NO: 11) (for driving expression, for example, in astrocytes), the glial fibrillary acidic protein (GFAP) promoter (SEQ ID NO: 14), the CMV promoter, and the Myo7A promoter. Additional promoters are contemplated herein including, but not limited to, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as the actin promoter, the myosin promoter, the elongation factor-1a promoter, the hemoglobin promoter, and the creatine kinase promoter. Additionally provided herein are a CBA promoter, the P546 MeCP2 promoter, hSyn promoter, hSST promoter, gfaABC(1)D promoter and GFAP promoter at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the respectively corresponding promoter nucleotide sequence of SEQ ID NOs: 9-14, which possess transcription promoting activity.

Examples of transcription control elements are tissue specific control elements, for example, promoters that allow expression specifically within neurons or specifically within astrocytes. Examples include neuron specific enolase and astrocyte-specific glial fibrillary acidic protein promoters. Inducible promoters are also contemplated. Non-limiting examples of inducible promoters include, but are not limited to a metallothionine promoter, a glucocorticoid promoter, a progesterone promoter, and a tetracycline-regulated promoter. The gene cassette may also include intron sequences to facilitate processing of a transgene RNA transcript when expressed in mammalian cells. One example of such an intron is the SV40 intron.

“Packaging” refers to a series of intracellular events that result in the assembly and encapsidation of an AAV particle. The term “production” refers to the process of producing the rAAV (the infectious, encapsidated rAAV particles) by the packing cells.

AAV “rep” and “cap” genes refer to polynucleotide sequences encoding replication and encapsidation proteins, respectively, of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses, baculoviruses and poxviruses such as vaccinia. The adenoviruses may encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). As described herein, “helper virus function” may be provided in a number of ways, including providing helper virus or providing, for example, polynucleotide sequences encoding the requisite function(s) to a producer cell in trans.

The rAAV genomes provided herein lack AAV rep and cap DNA. AAV DNA in the rAAV genomes (e.g., ITRs) contemplated herein may be from any AAV serotype suitable for deriving a recombinant virus including, but not limited to, AAV serotypes AAV1, AAV2, AAV3, AAV4, AAV5, AAV6, AAV7, AAV8, AAV9, AAV10, AAV11, AAV12, AAV13, AAVTT, Anc80, AAV-7M8, Anc80L65, AAVRH10, AAVRH74, and AAV-B1, and their derivatives. As noted above, the nucleotide sequences of the genomes of various AAV serotypes are known in the art. rAAV with capsid mutations, are also contemplated. See, for example, Marsic et al., Molecular Therapy, 22(11): 1900-1909 (2014). Modified capsids herein are also contemplated and include capsids having various post-translational modifications such as glycosylation and deamidation. Deamidation of asparagine or glutamine side chains resulting in conversion of asparagine residues to aspartic acid or isoaspartic acid residues, and conversion of glutamine to glutamic acid or isoglutamic acid is contemplated in rAAV capsids provided herein. See, for example, Giles et al., Molecular Therapy, 26(12): 2848-2862 (2018). Modified capsids herein are also contemplated to comprise targeting sequences directing the rAAV to the affected tissues and organs requiring treatment.

DNA plasmids provided herein comprise rAAV genomes described herein. The DNA plasmids may be transferred to cells permissible for infection with a helper virus of AAV (e.g., adenovirus, E1-deleted adenovirus or herpesvirus) for assembly of the rAAV genome into infectious viral particles with AAV9 capsid proteins. Techniques to produce rAAV, in which an rAAV genome to be packaged, rep and cap genes, and helper virus functions are provided to a cell are standard in the art. Production of rAAV particles requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs. Production of pseudotyped rAAV is disclosed in, for example, WO 01/83692 which is incorporated by reference herein in its entirety. AAV capsid proteins may be modified to enhance delivery of the recombinant rAAV. Modifications to capsid proteins are generally known in the art. See, for example, US 2005/0053922 and US 2009/0202490, the disclosures of which are incorporated by reference herein in their entirety.

A method of generating a packaging cell is to create a cell line that stably expresses all the necessary components for rAAV production. For example, a plasmid (or multiple plasmids) comprising a rAAV genome lacking AAV rep and cap genes, AAV rep and cap genes separate from the rAAV genome, and a selectable marker, such as a neomycin resistance gene, may be integrated into the genome of a cell. rAAV genomes may be introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line may then be infected with a helper virus such as adenovirus. The advantages of this method are that the cells are selectable and are suitable for large-scale production of rAAV. Other non-limiting examples of suitable methods employ adenovirus, herpesvirus or baculovirus rather than plasmids to introduce rAAV genomes and/or rep and cap genes into packaging cells.

General principles of rAAV particle production are reviewed in, for example, Carter, Current Opinions in Biotechnology, 1533-1539 (1992); and Muzyczka, Curr. Topics in Microbial. and Immunol., 158:97-129 (1992). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al., J. Virol., 62:1963 (1988); and Lebkowski et al., Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine 13:1244-1250 (1995); Paul et al., Human Gene Therapy, 4:609-615 (1993); Clark et al., Gene Therapy 3:1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV particle production.

Further provided herein are packaging cells that produce infectious rAAV particles. In one embodiment packaging cells may be stably transformed cancer cells such as HeLa cells, 293 cells and PerC.6 cells (a cognate 293 line). In another embodiment, packaging cells may be cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Also provided herein are rAAV (e.g., infectious encapsidated rAAV particles) comprising a rAAV genome of the disclosure. The genomes of the rAAV lack AAV rep and cap DNA, that is, there is no AAV rep or cap DNA between the ITRs of the genomes of the rAAV. The rAAV genome can be a self-complementary (sc) genome. A rAAV with a sc genome is referred to herein as a scAAV. The rAAV genome can be a single-stranded (ss) genome. A rAAV with a single-stranded genome is referred to herein as an ssAAV.

The rAAV may be purified by methods standard in the art such as by column chromatography and/or cesium chloride gradients. Methods for purifying rAAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

### Compositions

Compositions comprising rAAV are also provided. Compositions comprise a rAAV encoding a polypeptide of interest including, but not limited to, a GAT-1 protein. Compositions may include two or more rAAV encoding different polypeptides of interest.

Compositions provided herein comprise rAAV and a pharmaceutically acceptable excipient or excipients. Acceptable excipients are nontoxic to recipients and are preferably inert at the dosages and concentrations employed, and include, but are not limited to, buffers such as phosphate e.g., phosphate-buffered saline (PBS), citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, copolymers such as poloxamer 188, pluronics (e.g., Pluronic F68) or polyethylene glycol (PEG). Compositions provided herein can comprise a pharmaceutically acceptable aqueous excipient containing a non-ionic, low-osmolar compound or contrast agent such as iobitridol, iohexol, iomeprol, iopamidol, iopentol, iopromide, ioversol, or ioxilan, where the aqueous excipient containing the non-ionic, low-osmolar compound can have one or more of the following characteristics: about 180 mgl/mL, an osmolality by vapor-pressure osmometry of about 322 mOsm/kg water, an osmolarity of about 273 mOsm/L, an absolute viscosity of about 2.3 cp at 20° C. and about 1.5 cp at 37° C., and a specific gravity of about 1.164 at 37° C. Exemplary compositions comprise about 20 to 40% non-ionic, low-osmolar compound or about 25% to about 35% non-ionic, low-osmolar compound. An exemplary composition comprises scAAV or rAAV viral particles formulated in 20 mM Tris (pH8.0), 1 mM MgCl₂, 200 mM NaCl, 0.005% poloxamer 188 and about 25% to about 35% non-ionic, low-osmolar compound. Another exemplary composition comprises scAAV formulated in and 1×PBS and 0.001% Pluronic F68.

For CSF delivery including but not limited to intrathecal delivery, the viral vector can be mixed with a contrast agent (Omnipaque or similar). For example, the compositions may comprise a non-ionic, low-osmolar contrast agent including, but not limited to, iobitridol, iohexol, iomeprol, iopamidol, iopentol, iopromide, ioversol, ioxilan, or combinations thereof.

Dosages may be expressed in units of viral genomes (vg). Dosages contemplated herein include about 1×10⁷ vg, about 1×10⁸ vg, about 1×10⁹ vg, about 5×10⁹ vg, about 6×10⁹ vg, about 7×10⁹ vg, about 8×10⁹ vg, about 9×10⁹ vg, about 1×10¹⁰ vg, about 2×10¹⁰ vg, about 3×10¹⁰ vg, about 4×10¹⁰ vg, about 5×10¹⁰ vg, about 1×10¹¹ vg, about 1.1×10¹¹ vg, about 1.2×10¹¹ vg, about 1.3×10¹¹ vg, about 1.2×10¹¹ vg, about 1.3×10¹¹ vg, about 1.4×10¹¹ vg, about 1.5×10¹¹ vg, about 1.6×10¹¹ vg, about 1.7×10¹¹ vg, about 1.8×10¹¹ vg, about 1.9×10¹¹ vg, about 2×10¹¹ vg, about 3×10¹¹ vg, about 4×10¹¹ vg, about 5×10¹¹ vg, about 1×10¹² vg, about 1×10¹³ vg, about 1.1×10¹³ vg, about 1.2×10¹³ vg, about 1.3×10¹³ vg, about 1.5×10¹³ vg, about 2×10¹³ vg, about 2.5×10¹³ vg, about 3×10¹³ vg, about 3.5×10¹³ vg, about 4×10¹³ vg, about 4.5×10¹³ vg, about 5×10¹³ vg, about 6×10¹³ vg, about 1×10¹⁴ vg, about 2×10¹⁴ vg, about 3×10¹⁴ vg, about 4×10¹⁴ vg, about 5×10¹⁴ vg, about 1×10¹⁵ vg, to about 1×10¹⁶ vg, or more total viral genomes. Dosages of about 1×10⁹ vg to about 1×10¹⁰ vg, about 5×10⁹ vg to about 5×10¹⁰ vg, about 1×10¹⁰ vg to about 1×10¹¹ vg, about 1×10¹¹ vg to about 1×10¹⁵ vg, about 1×10¹² vg to about 1×10¹⁵ vg, about 1×10¹² vg to about 1×10¹⁴ vg, about 1×10¹³ vg to about 6×10¹⁴ vg, and about 6×10¹³ vg to about 1.0×10¹⁴ vg, 2.0×10¹⁴ vg, 3.0×10¹⁴ vg, 5.0×10¹⁴ are also contemplated. One dose exemplified herein is1.65×10¹¹ vg.

For example, CSF doses can range between about 1×10¹³ vg/patient to about 1×10¹⁵ vg/patient based on age groups. For example, intravenous delivery doses can range between 1×10¹³ vg/kilogram (kg) body weight and 2×10¹⁴ vg/kg.

### Methods of Treatment

Methods of treatment herein target cells with reduced GAT-1 protein GABA transporter activity. Methods of treatment herein can target cells with a “defective” SLC6A1 gene, that is a gene with at least one “defective” (i.e., mutated) allele encoding a GAT-1 protein that lacks GABA transporter activity. As is understood in the art, a diploid subject such as a human subject generally has two copies of each gene which are referred to alleles. Methods of transducing target such cells in a subject (e.g., a human subject) are provided. Methods of transducing, for example, one or more of neurons, astrocytes and/or central nervous system tissue in a subject (e.g., a human subject) are provided.

Use of methods of treatment described herein is indicated for reduced GAT-1 protein GABA transporter activity in, for example, epileptic encephalopathy such as pediatric epileptic encephalopathy. The methods increase GAT-1 mRNA and protein expression levels.

The terms “transducing” and “transduction” are used to refer to the administration/delivery of rAAV of the disclosure encoding a GAT-1 protein with GABA transporter activity to a target cell either in vivo or in vitro, resulting in expression of a functional GAT-1 protein by the target cell. Transduction of cells with rAAV of the disclosure results in sustained expression of polypeptide encoded by the rAAV.

Methods provided herein transduce target cells with one or more rAAV described herein. In some embodiments, the rAAV viral particle comprising a transgene is administered or delivered to the CSF of a subject by, for example, by intracerebroventricular injection, cisternal injection or lumbar intrathecal injection, or other injection method(s) accessing the CSF, or via intravenous delivery, or via a combination of such routes. Intrathecal administration refers to delivery into the space under the arachnoid membrane of the brain or spinal cord. Intrathecal administration to the brain in particular can by carried out by intracerebroventricular injection. Areas of the brain contemplated for delivery include, but are not limited to, the motor cortex, visual cortex, cerebellum and the brain stem.

For intrathecal administration, the subject can be held in the Trendelenburg position (head down position) after injection of the rAAV (e.g., for about 5, about 10, about 15 or about 20 minutes). For example, the patient may be tilted in the head down position at about 1 degree to about 30 degrees, about 15 to about 30 degrees, about 30 to about 60 degrees, about 60 to about 90 degrees, or about 90 to about 180 degrees.

The treatment methods provided herein comprise the step of administering an effective dose, or effective multiple doses, of a composition comprising a rAAV provided herein to a subject (e.g., an animal including, but not limited to, a human patient) in need thereof. If the dose is administered prior to development of symptoms, the administration is prophylactic. If the dose is administered after the development of symptoms, the administration is therapeutic. An effective dose is a dose that alleviates (eliminates or reduces) at least one symptom associated with a condition of reduced level of GAT-1 GABA transporter activity, that slows or prevents progression of the condition, that diminishes the extent of the condition, that results in remission (partial or total) of the condition, and/or that prolongs survival.

An effective dose for treatment of epileptic encephalopathy is a dose that alleviates (eliminates or reduces) seizures, impaired cognitive development, and ataxia.

### EXAMPLES

While the following examples describe specific embodiments, variations and modifications will occur to those skilled in the art. Accordingly, only such limitations as appear in the claims should be placed on the invention.

### Example 1

### Production of rAAV Expressing GAT-1

rAAV were produced using transgenes expressing a human SLC6A1 cDNA (SEQ ID NO: 1) (Origene Catalog #SC126769) for use in a gene therapy re-expression approach for SLC6A1 pathology. At 4494 base pairs, SLC6A1 fits within the confines of AAV.

AAV9 primarily targets neurons and secondarily astrocytes. Expression level and cell type specific transduction are further tailored by using different promoters to drive transgene expression. The first construct yields strong, ubiquitous expression by driving SLC6A1 expression with a CAG promoter. CAG was selected over other ubiquitous promoters, such as CBA, due to its strong bias towards inhibitory neurons compared to excitatory neurons [Nathanson et al., Neuroscience, 161(2):441-450 (2009)]. Next, a truncated version of the MECP2 promoter, known as p546, is used to target neurons and astrocytes but with a reduced potency. A third construct limits expression to neurons by use of the synapsin promoter (Nathanson, Neuroscience, supra) and a somatostatin promoter further limits expression to inhibitory GABAergic neurons [Nagai et al., Biochem Biophys Res Commun., 518(4):619-624 (2019); Nathanson et al., Front Neural Circuits., 3:19 (2009)]. Lastly, GFAP is used to target astrocytes [Lawlor et al., Mol Ther., 17(10):1692-1702 (2009)]. After cloning, the five constructs were sequenced, expression was confirmed in 293 cells, and small scale AAV9 viral preparations were generated.

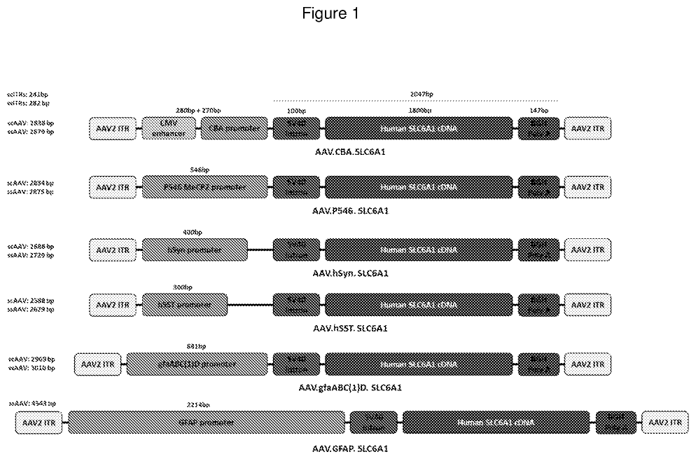

The transgenes shown in FIG. 1 use the foregoing promoters to drive expression of the GAT-1 protein from the human SLC6A1 cDNA. The transgene of SEQ ID NO: 3 includes the CBA promoter (a ubiquitous promoter), the transgene of SEQ ID NO: 4 includes the P546 MeCP2 promoter (a ubiquitous promoter), the transgene of SEQ ID NO: 6 includes the hSyn promoter (a neuron-specific promoter), the transgene of SEQ ID NO: 7 includes the hSST promoter (an inhibitory neuron-specific promoter), the transgene of SEQ ID NO: 5 includes the gfaABC(1)D promoter (an astrocyte-specific promoter) and the transgene of SEQ ID NO: 8 includes the GFAP promoter (an astrocyte-specific promoter). Promoters contemplated for use in methods herein are of a size that allow the transgene constructs to fit well in rAAV and also drive transgene expression to increase GAT-1 protein expression by at least 10% in transduced cells, focusing on increasing expression in neurons and astrocytes.

The transgenes were subcloned into AAV9 production plasmids namely, pscAAV.SLC6A1.CBA, pscAAV.SLC6A1.P546, pscAAV.SLC6A1.hSyn, pscAAV.SLC6A1.SST, pscAAV.SLC6A1.gfaABC(1)D, pssAAV.SLC6A1.GFAP, and scAAV and ssAAV were produced as described in Foust et al., Nat Biotechnol., 27(1): 59-65 (2009) by transient triple transfection of 293 cells using the double-stranded AAV2-ITR-based production plasmids, a plasmid encoding Rep2Cap9 sequence and an adenoviral helper plasmid pHelper.

Resulting SLC6A1 rAAV were named ssAAV.CBA.SLC6A1, ssAAV.P546.SLC6A1, ssAAV.hSyn.SLC6A1, ssAAV.hSST.SLC6A1, ssAAV.gfaABC(1)D.SLC6A1, SSAAA.GFAP.SLC6A1, scAAV.CBA.SLC6A1 (also sometimes referred to herein as scAAV.CAG.SLC6A1), scAAV.P546.SLC6A1, scAAV.hSyn.SLC6A1 (also sometimes referred to herein as scAAV.Syn.SLC6A1 herein), scAAV.hSST.SLC6A1 (also sometimes referred to herein as scAAV.SST.SLC6A1 herein), scAAV.gfaABC(1)D.SLC6A1 and scAAA.GFAP.SLC6A1.

### Example 2

### In Vitro Expression Analysis

Expression of SLC6A1 mRNA from the AAV production plasmids generated in Example 1 was evaluated in HEK293T cells. HEK293T cells were transfected with one of the AAV production plasmids expressing SLC6A1 cDNA or a control scAAV expressing green fluorescent protein (GFP). Cells were harvested 72 hours post-transfection and SLC6A1 mRNA expression was analyzed by PCR and qPCR.

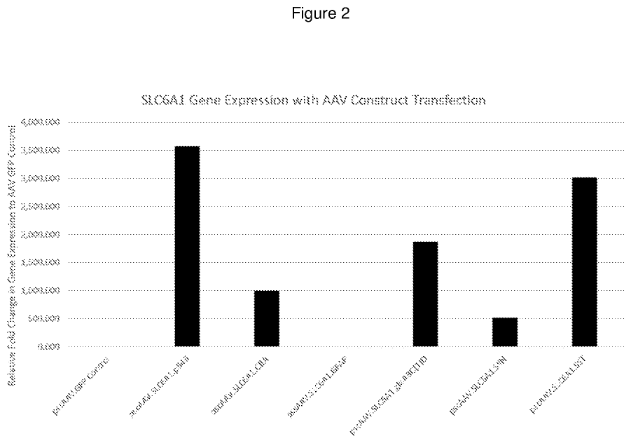

SLC6A1 mRNA expression levels are shown in FIG. 2 as relative fold change to control GFP mRNA expression levels.

### Example 3

### Expression in Wild Type Mice

Expression of SLC6A1 mRNA from the scAAV and ssAAV generated in Example 1 was evaluated in wild type mice. Each of the AAV9 constructs expressing SLC6A1 was administered to four newborn wild type mice (n=4 for each rAAV9) by intracerebroventricular injection of 2.95×10¹⁰-1.5×10¹¹ vg/mouse. WT mice were sacrificed at ˜four weeks (short-term) or ˜four months (long-term) of age along with an uninjected WT control mouse. Tissues were harvested for SLC6A1 mRNA expression, protein expression, and histology.

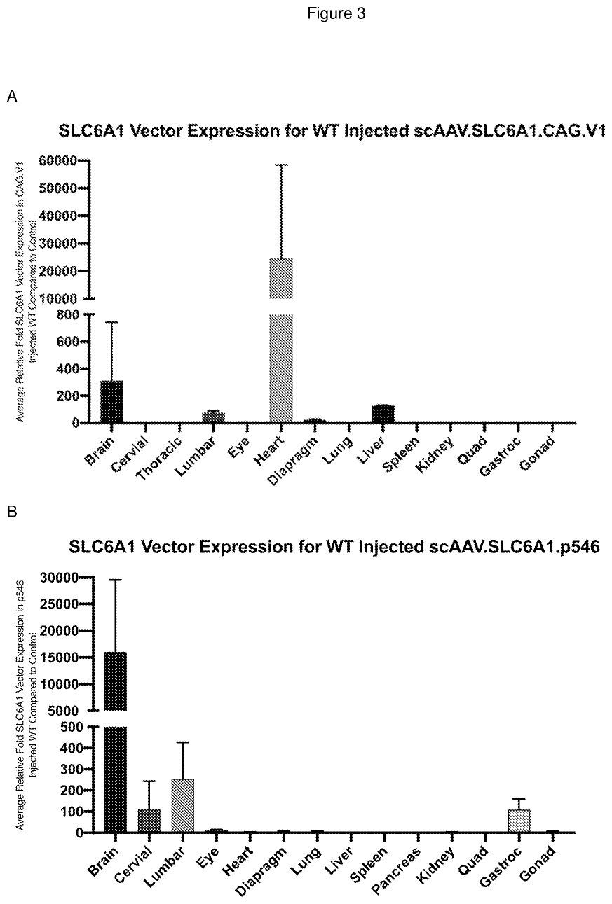

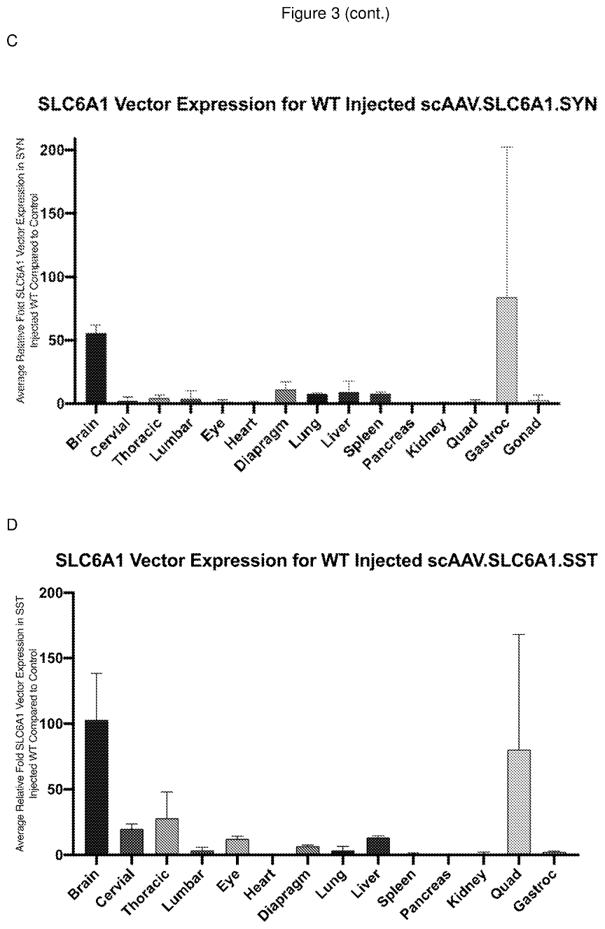

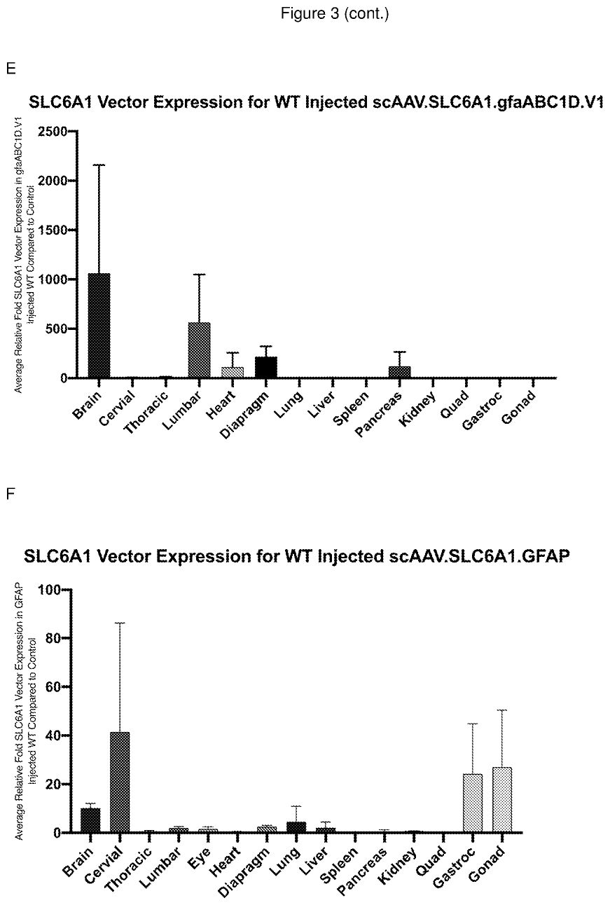

SLC6A1 mRNA expression levels in injected mice are shown in FIG. 3A-E as relative fold change to control uninjected, age-matched mice mRNA expression levels. There were no sign of toxicity clinically or on bloodwork.

### Example 4

### Administration of AAV Gene Therapy Constructs in Murine Models of SLC6A1

While a GAT-1 knockout mouse exists [Jensen et al., J Neurophysiol, 90(4): 2690-2701 (2003) and Chiu et al., J Neurosci, 22(23): 10251-10266 (2002], heterozygous mice have no phenotype and thus do not recapitulate disease in human patients. Homozygous mice do exhibit behavioral and cognitive deficits and seizures beginning at postnatal day 19 which can be quantified by EEG. Two additional mouse models have missense mutations A288V and S295L, which are equivalent to known human point mutations. Unlike the knockout mouse, both A288V and S295L mice are phenotypically abnormal as heterozygotes, including the presence of seizures. A288V and S295L murine models of SLC6A1 are useful to evaluate safety and efficacy of gene replacement strategies.

All viral vectors were evaluated using the S295L mouse model. The effects of SCAAV.P546.SLC6A1, scAAV.hSyn.SLC6A1, scAAV.hSST.SLC6A1, scAAV.gfaABC(1)D.SLC6A1 (with gfaABC(1)D.v2 transgene, SEQ ID NO: 5) and ssAAA.GFAP.SLC6A1were compared to control empty viral particles. Each of the rAAV9 was administered to newborn mice by intracerebroventricular injection of 3×10¹⁰ vg/mouse.

Mice underwent various behavioral tests including weekly weight, bi-weekly rotarod, cage hang and clasping tests starting at 21-28 days. At a humane or predetermined time point, the animals were sacrificed for post-mortem biochemical, molecular, and histological analyses, including transgene expression analyses.

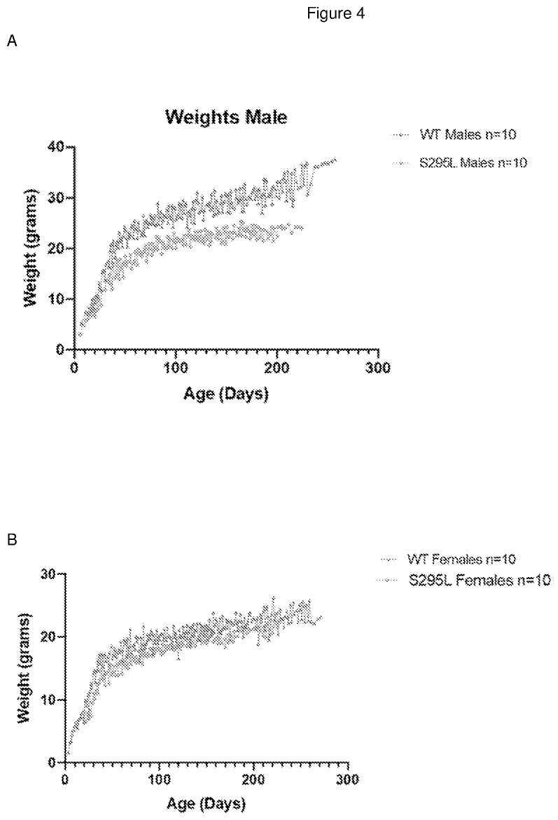

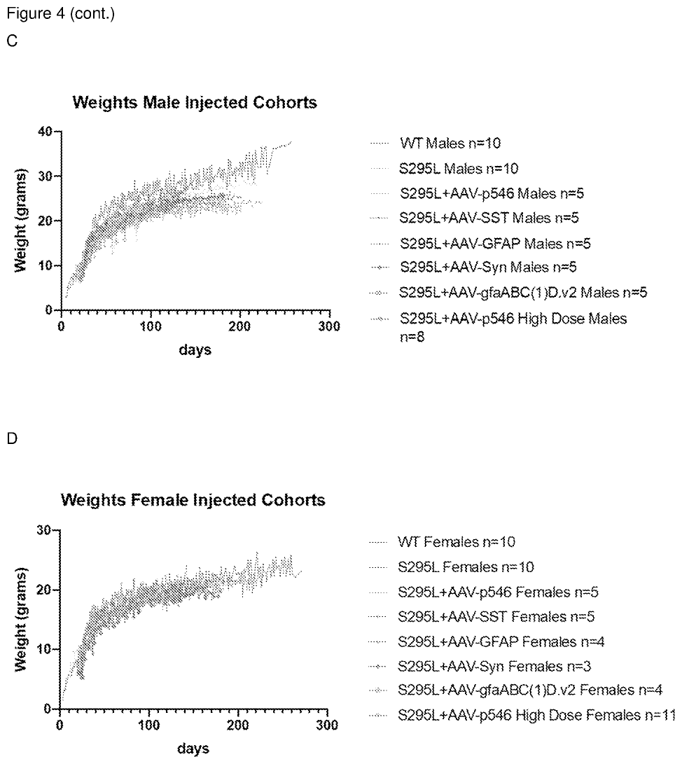

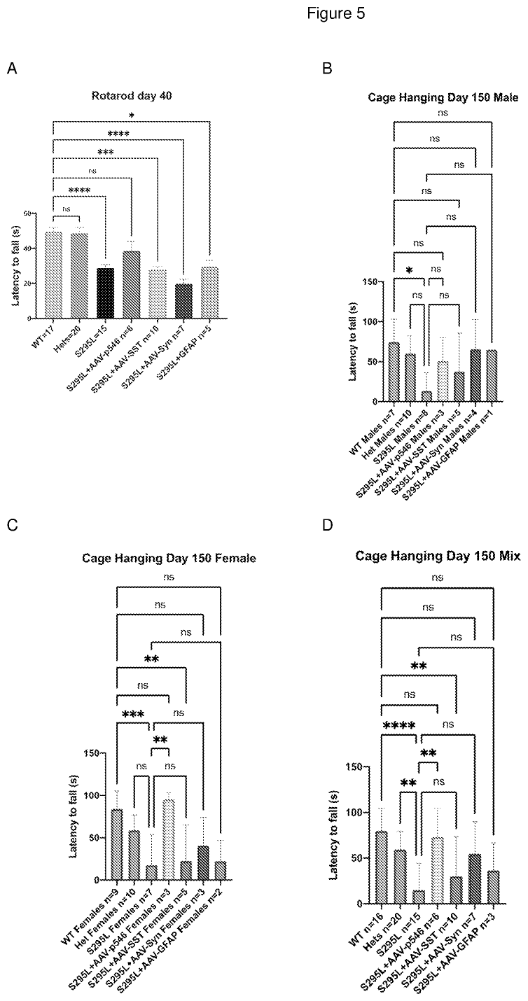

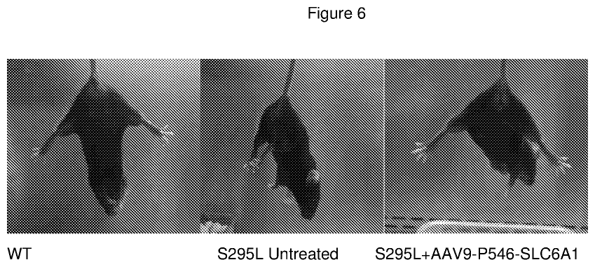

Results are shown in FIG. 4A-D (weight), FIG. 5A-C (rotarod and cage hang) and FIG. 6 (clasping).

Weight development in males and females was compared between wild type animals, heterozygous animals and homozygous animals containing the S295L mutation in the SLC6A1 gene. Untreated homozygous mutant male mice showed reduced weight gain. Importantly, the weight gain in the homozygous mutant male mice was normalized when they were treated with AAV-p546-SLC6A1 (i.e., scAAV.P546.SLC6A1). No differences were seen in female homozygous mutant mice that were treated with any construct compared to wild type animals, but that was to be expected since there is no difference between untreated females and wild type females either. See, FIG. 4A-D.

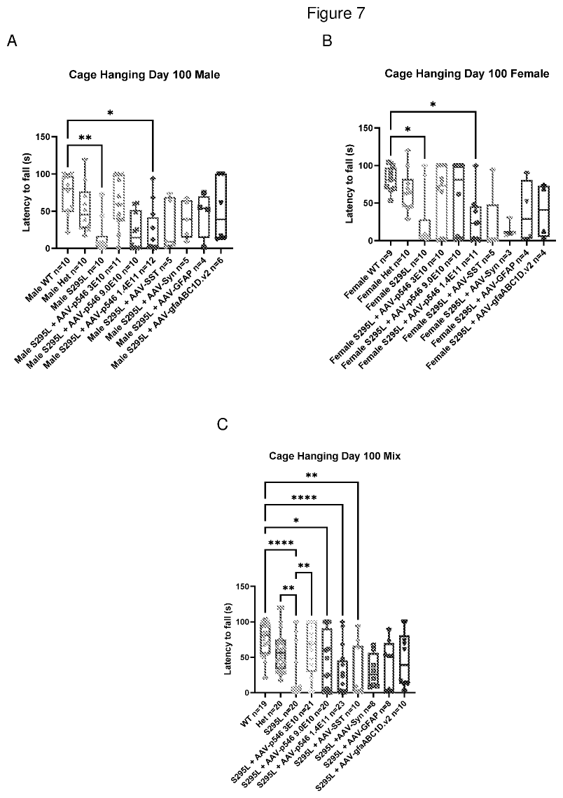

In the rotarod assays performed, mice were placed on a rotating wheel that continues to turn in accelerated manner. The time until the mice fall off was measured. Highly significant differences were seen in the ability to remain on the rotarod between wild type animals versus homozygous mutant animals at 40 days of age. In addition, wire hang tests were performed in which the mice are placed on a cage lid containing a metal wire and then the lid is turned upside down. The time the animals are able to hang onto the grid before they fall off is measured. Very significant differences at 150 days were found in both males and females. At that point, homozygous mutant mice showed highly reduced cage hanging ability. The different AAV-SLC6A1 constructs had various effects on improving the ability of the mutant mice to hang for a longer period of time. The most effective construct was AAV-P546-SLC6A1 (i.e., scAAV.P546.SLC6A1), which reduced the latency to fall to normal wild type levels. See, FIG. 5A-C. FIG. 7A-C shows data from additional mice.

Untreated homozygote mutant mice display a clasping phenotype in which they are unable to properly spread out the hindlimbs when picked up by the tail. As shown in FIG. 6, wild type mice (left image) and treated mutant mice were indistinguishable (right image) and were both showing normal hindlimb positioning in this assay.

### Example 5

### Quantification of Epileptiform Discharges in Mice

Since seizure activity is a common disease phenotype in SLC6A1 subjects, the mice above were also tested by electroencephalogram (EEG).

Mice were premedicated using injectable Buprenorphine HCL at 1 mg/kg and Carprofen at 5 mg/kg. Animals were then induced using isoflurane via an induction chamber. Anesthetic maintenance was performed using 1-3% isoflurane via a nose cone. Once animals reached a surgical plane of anesthesia the surgical site was aseptically prepped. An ˜2.5 cm long incision was made cranially from the shoulder blades extending just caudal to the base of the eyes. The DSI telemetry implant was inserted subcutaneously. Biopotential leads were surgically placed in the trapezius muscle for EMG monitoring. Additionally, drill holes were made into the skull and electrodes were placed in the following coordinates using a stereotaxic device for EEG monitoring: AP+1.0/ML−1.5 (LH) and AP−2.0/ML+2.0 (RH). The incision was closed using a simple continuous suture pattern.

Postoperatively mice were given 1mL of warm NaCL, administered Capromorelin at 2 mg/kg orally, and placed in an incubator for at least 12 hours. Mice were allowed to heal for at least 72 hours and maintained on oral Carpofen through their drinking water. Data was then acquired by placing the mice directly on the wireless telemetry receiver plate system for 24 hours. Data was then analyzed in the DSI NeuroScore™ software.

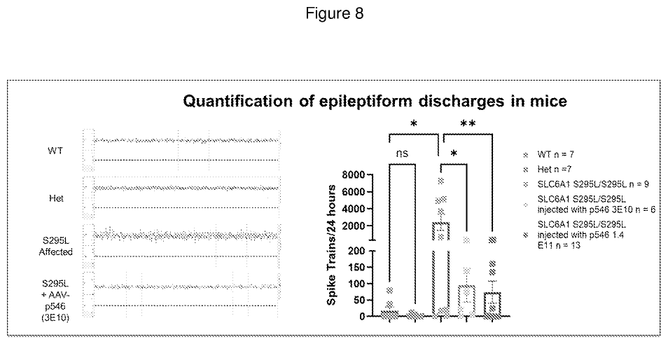

Results are shown in FIG. 8. Homozygote mutant mice exhibited a significant increase in spike trains/24 hours as compared to heterozygote control mice. Homozygote mutant mice treated with AAV9-P546-SLC6A1 (i.e., scAAV.P546.SLC6A1) exhibited a significant reduction in spike trains/24 hours when compared to untreated homozygote mutant mice.

### Example 6

### AAV Gene Therapy for Human Patients

AAV gene therapy provides to patients a wildtype copy of SLC6A1 in order to address the haploinsufficiency resultant from mutations in one copy of the SLC6A1 gene.

While the present invention has been described in terms of specific embodiments, it is understood that variations and modifications will occur to those skilled in the art. Accordingly, only such limitations as appear in the claims should be placed on the invention.

All documents referred to in this application are hereby incorporated by reference in their entirety.

What is claimed:

1. A method of delivering a transgene encoding a GAT-1 protein with GABA transporter activity to a subject in need of GAT-1 protein GABA transporter activity,
wherein the method comprises administering a gene therapy vector comprising the transgene to the cerebrospinal fluid of a subject by intracerebroventricular injection, cisternal injection, lumbar intrathecal injection, or by intravenous delivery; and
wherein the transgene comprises the polynucleotide of SEQ ID NO: 1, or a polynucleotide at least 90%, 91%, 92%, 93%, 94%, 95%, 96%, 97%, 98%, or 99% identical to SEQ ID NO: 1 that encodes a GAT-1 protein with GABA transporter activity; or the transgene comprises the polynucleotide of SEQ ID NO: 3, 4, 5, 6, 7, or 8, or a polynucleotide at least 90%, 91%, 92%, 93%, 94%, 95%, 96%, 97%, 98%, or 99% identical to SEQ ID NO: 3, 4, 5, 6, 7, or 8 that encodes a GAT-1 protein with GABA transporter activity.

2. The method of claim 1 wherein the subject has at least one defective SLC6A1allele.

3. The method of claim 1 or 2 wherein the transgene is delivered to neurons or astrocytes, or both, of the subject.

4. The method of any one of claims 1-3 wherein the administration treats at least one of seizures, impaired cognitive development and ataxia in the subject.

5. The method of any one of claims 1-4 wherein the administration treats epileptic encephalopathy in the subject.

6. The method of claim 5 wherein the epileptic encephalopathy is pediatric epileptic encephalopathy.

7. The method of any one of claims 1-6 wherein the gene therapy vector is an AAV1, AAV2, AAV3, AAV4, AAV5, AAV6, AAV7, AAV8, AAV9, AAV10, AAV11, AAV12, AAV13, AAVTT, Anc80, AAV-7m8, Anc80L65, AAVRH10, AAVRH74, or AAV-B1, or a derivative of any thereof.

8. The method of any one of claims 1-7, wherein the gene therapy vector is administered by intrathecal delivery.

9. The method of any one of claims 1-8, wherein the gene therapy vector is administered by intrathecal delivery, and the method further comprises placing the subject in the Trendelenburg position after administration of the gene therapy vector.

10. The method of any one of claims 1-7, wherein the gene therapy vector is administered by intracerebroventricular injection.

11. The method of any one of claims 1-7, wherein the gene therapy vector is administered by cisternal injection.

12. The method of any one of claims 1-7, wherein the gene therapy vector is administered by lumbar intrathecal injection.

13. The method of any one of claims 1-7, wherein the gene therapy vector is administered by intravenous delivery.

14. A transgene comprising the polynucleotide of SEQ ID NO: 1, or a polynucleotide at least 90%, 91%, 92%, 93%, 94%, 95%, 96%, 97%, 98%, or 99% identical to SEQ ID NO: 1 that encodes a GAT-1 protein with GABA transporter activity.

15. A transgene comprising the polynucleotide of SEQ ID NO: 3, 4, 5, 6, 7, or 8, or a polynucleotide at least 90%, 91%, 92%, 93%, 94%, 95%, 96%, 97%, 98%, or 99% identical to SEQ ID NO: 3, 4, 5, 6, 7, or 8 that encodes a GAT-1 protein with GABA transporter activity.

16. A recombinant adeno-associated virus (rAAV) with a genome comprising a transgene comprising the polynucleotide of SEQ ID NO: 1, or a polynucleotide at least 90%, 91%, 92%, 93%, 94%, 95%, 96%, 97%, 98%, or 99% identical to SEQ ID NO: 1 that encodes a GAT-1 protein with GABA transporter activity.

17. A recombinant adeno-associate virus (rAAV) with a genome comprising a transgene of SEQ ID NO: 3, 4, 5, 6, 7, or 8, or a polynucleotide at least 90%, 91%, 92%, 93%, 94%, 95%, 96%, 97%, 98%, or 99% identical to SEQ ID NO: 3, 4, 5, 6, 7, or 8 that encodes a GAT-1 protein with GABA transporter activity.

18. The recombinant adeno-associated virus (rAAV) of claim 16 or 17 wherein the recombinant adeno-associate virus is an AAV1, AAV2, AAV3, AAV4, AAV5, AAV6, AAV7, AAV8, AAV9, AAV-10, AAV11, AAV12, AAV13, AAVTT, Anc80, AAV-7m8, Anc80L65, AAVRH10, AAVRH74, or AAV-B1 serotype vector, or a derivative thereof.

19. The recombinant adeno-associate virus (rAAV) of claim 18 that is an AAV9.
